(19) United States
(12) Patent Application Publication
(10) Pub. No.: US 2021/0290961 A1
(43) Pub. Date: Sep. 23, 2021

(54) SYSTEMS FOR TREATING OBSTRUCTIVE SLEEP APNEA

(71) Applicant: THE ALFRED E. MANN FOUNDATION FOR SCIENTIFIC RESEARCH, VALENCIA, CA (US)

(72) Inventors: Robert J. Greenberg, LOS ANGELES, CA (US); HARSHIT R. SURI, PASADENA, CA (US); JOSEPH L. CALDERON, SANTA CLARITA, CA (US); BRIAN R. DEARDEN, PASADENA, CA (US)

(21) Appl. No.: 17/210,033

(22) Filed: Mar. 23, 2021

Related U.S. Application Data
(60) Provisional application No. 62/993,580, filed on Mar. 23, 2020.

(51) Int. Cl. A61N 1/36; A61N 1/372

(52) U.S. Cl. CPC A61N 1/36139; A61N 1/3611; A61N 1/36167; A61N 1/3615; A61N 1/37217

(57) ABSTRACT

Systems for treating obstructive sleep apnea having an implanted stimulator with an internal sensor configured to generate sensory data corresponding to movement of the thoracic or abdominal cavity of a patient during respiration. The system includes a wireless communications link between the stimulator and at least one external sensor for sensing a patient's physiological parameter and is used to augment the sensory data from the internal sensor. The stimulator includes a stimulation system configured to deliver electrical stimulation to a nerve which innervates an upper airway muscle, such as the hypoglossal nerve to treat sleep apnea.

### CROSS-REFERENCE TO RELATED APPLICATION

This application claims the benefit of U.S. Provisional Application Ser. No. 62/993,580, entitled “SYSTEMS FOR TREATING OBSTRUCTIVE SLEEP APNEA” and filed on Mar. 23, 2020, which is expressly incorporated by reference herein in its entirety.

### BACKGROUND

Obstructive Sleep Apnea (OSA) is a sleep disorder involving obstruction of the upper airway during sleep. The obstruction of the upper airway may be caused by the collapse of or increase in the resistance of the pharyngeal airway, often resulting from tongue obstruction. The obstruction of the upper airway may be caused by reduced genioglossus muscle activity during the deeper states of NREM sleep. Obstruction of the upper airway may cause breathing to pause during sleep. Cessation of breathing may cause a decrease in the blood oxygen saturation level, which may eventually be corrected when the person wakes up and resumes breathing. The long-term effects of OSA include high blood pressure, heart failure, strokes, diabetes, headaches, and general daytime sleepiness and memory loss, among other symptoms.

OSA is extremely common, and may have a prevalence similar to diabetes or asthma. Over 100 million people worldwide suffer from OSA, with about 25% of those people being treated. Continuous Positive Airway Pressure (CPAP) is a conventional therapy for people who suffer from OSA. More than five million patients own a CPAP machine in North America, but many do not comply with use of these machines because they cover the mouth and nose and, hence, are cumbersome and uncomfortable.

Neurostimulators may be used to open the upper airway as a treatment for alleviating apneic events. Such therapy may involve stimulating the nerve fascicles of the hypoglossal nerve (HGN) that innervate the intrinsic and extrinsic muscles of the tongue in a manner that prevents retraction of the tongue which would otherwise close the upper airway during the inspiration period of the respiratory cycle. ImThera Medical has been in FDA clinical trials for a stimulator system that is used to stimulate the trunk of the HGN with a nerve cuff electrode. The stimulation system does not provide a sensor or sensing, and therefore, the stimulation delivered to the HGN trunk is not synchronized to the respiratory cycle. Thus, the tongue and other muscles that are innervated by nerve fascicles of the HGN trunk are stimulated irrespective of the respiratory cycle.

The rationale for this treatment method appears to be that it is enough simply to tone the tongue muscle and other nearby muscles, so that the tongue muscle does not retract in a manner that would cause OSA. The belief is that it is not necessary to specifically target the protraction (i.e., anterior movement) of the tongue muscle and to synchronize the occurrence of tongue protraction when it is most needed, i.e., during inspiration. The nerve cuff electrode of the ImThera Medical system has multiple electrode contacts helically surrounding the proximal part of the HGN nerve trunk. So, instead, each electrode contact delivers stimulation in a sequential order to the HGN trunk. For example, if a three-electrode contact nerve cuff is used, electrode contact #1 stimulates, then stops, electrode contact #2 stimulates, then stops, electrode contact #3 stimulates, then stops, then electrode contact #1 stimulates, then stops and so on. Since all or most electrode contacts deliver stimulation, there is no selection process to choose the best one or two electrode contacts that is finally used to deliver the best stimulation to alleviate sleep apnea.

A disadvantage of the ImThera Medical system is that it does not target tongue protraction coincident with the inspiration phase of respiration, since it does not have a sensor to enable synchronized stimulation of the respiratory cycle. Since there is no attempt to synchronize the stimulation with the respiratory cycle, the tongue protraction does not occur when it would appear to help the most—during inspiration when OSA can occur. Also, because the HGN trunk contains nerve fascicles that innervate muscles other than the muscle that extend the tongue, the Imthera Medical method of stimulation at the HGN trunk does not just target the specific protrusor muscles of the tongue muscle, but other muscles that are not targeted. Thus, stimulating the HGN trunk in an arbitrary manner may recruit other nerve fascicles of the HGN trunk that may not contribute to the protraction of the tongue.

Another company, Inspire Medical Systems, Inc., does offer a stimulation system with a sensor, and therefore does attempt to time the onset of stimulation to the breathing cycle. This system, which is FDA approved for sale in the United States since April 2010, uses a simple, bipolar electrode (two electrode contacts only) within a nerve cuff electrode and implants the electrode at the branch of the HGN that is responsible for protruding the tongue. A simple, two-electrode contact or three-electrode contact cuff electrode can be used at the branch nerve, unlike the HGN trunk, because at the distal branch location, the nerve fascicles generally innervate the specific tongue protrusor muscle and not other muscles.

However, implanting the electrode at a branch of the HGN requires additional surgery time, which increases trauma to the patient and increases the substantial expense of operating room time. By attaching the nerve cuff electrode to the proximal section of the main trunk of the HGN, compared to placing the nerve cuff electrode at the more distal end of the HGN, the surgical time may be reduced by approximately one hour or more. Further, because the branch nerve is small and more difficult to isolate than the HGN trunk, implanting a nerve cuff electrode at the branch site demands heightened expertise from the otolaryngologist/Ear Nose and Throat (ENT) surgeon or neurosurgeon, which may increase the chance for error and surgical risks. Furthermore, because the distal location of the HGN has a smaller diameter of nerves, and hence the required electrodes need to be smaller, the smaller nerve cuff electrode may be more difficult to manufacture.

Thus, it is certainly desirable to implant the nerve cuff electrode at the trunk of the hypoglossal nerve. However, one must then deal with the fact that the target nerve fascicles may be near the center of the nerve trunk and are not easily isolated and stimulated, while at the same time avoiding stimulating other non-targeted fascicles in the same nerve trunk.

Furthermore, a pressure sensor is connected to neurostimulator of the Inspire system by a lead, thereby allowing the pressure sensor to be placed remotely from the implanted site of the neurostimulator. However, the fact that the pressure sensor has a lead connected to the stimulator necessitates some additional surgery, because the sensor lead is another appendage that must be implanted.

There, thus, remains a need for improved systems and methods for selectively recruiting only the fascicles of the hypoglossal nerve in synchronization with the respiratory cycle for treating OSA of a patient, while minimizing the surgery time and effort required to implant the neurostimulation components in the patient.

### BRIEF SUMMARY OF EXEMPLARY ASPECTS OF THE DISCLOSURE

The following presents a simplified summary of one or more aspects in order to provide a basic understanding of such aspects. This summary is not intended as an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

One aspect of the present disclosure relates to a system for treating obstructive sleep apnea. The system comprises an implanted stimulator with an internal sensor configured to generate sensory data corresponding to movement of the thoracic or abdominal cavity of a patient during respiration. The system includes a wireless communications link between the stimulator and an external sensor, which generates sensory data corresponding to one or more of a patient's physiological parameters and is used to augment the sensory data from at least one internal sensor. The stimulator includes a stimulation system configured to deliver electrical stimulation to a nerve which innervates an upper airway muscle, such as the hypoglossal nerve to treat sleep apnea. The stimulator has a controller coupled to one in more internal and/or external (e.g., external) sensors and the stimulation system. The controller may be configured to cause the stimulation system to stimulate the nerve based on the sensory data received from one or more of the internal and/or external sensors.

Previously designed implantable systems to treat OSA (e.g., the Inspire, Imthera, Apnex, Cyberonix systems) have relied on implantable stimulators and implantable sensing (except for Imthera). An implantable device with a battery (and embedded sensing modalities) poses a challenge in terms of battery life management and upgrading firmware in the device. Also, having a sensor in the implanted device increases risk of the device not adequately detecting respiratory artifacts across patients with various anatomical variations. This could result in reduction in the efficacy of therapy. The use of external sensors decoupled from the implanted system allows for a wide range of sensors to be used to detect respiration artifacts for example RIP-bands (Respiratory Inductance Plethysmography) integrated into the harness, inertial sensors, pressure sensors, audio sensors, peripheral oxygen saturation (SpO₂) sensors (e.g., an LED-based SpO₂ sensor), etc. Moreover, the external sensors can now be placed in various anatomical locations, which might not have been accessible had the sensor been implanted. These and other advantages of such systems are described in further detail bellow.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 is a diagram illustrating an exemplary embodiment of a system for treating obstructive sleep apnea.

FIG. 2A is a diagram illustrating an exemplary embodiment of a system for treating obstructive sleep apnea with a finger-mounted external sensor.

FIG. 2B is a diagram illustrating an exemplary embodiment of a system for treating obstructive sleep apnea with a head-mounted external sensor.

FIG. 2C is a diagram illustrating an exemplary embodiment of a system for treating obstructive sleep apnea with a chest-mounted external sensor (e.g., held in proximity to the chest via a harness).

FIG. 3 is a diagram illustrating an exemplary embodiment of an electroencephalogram (EEG) sensor shaped and configured for positioning behind the ear of a patient.

FIG. 4 is a block diagram of an exemplary embodiment of a system for treating obstructive sleep apnea.

FIG. 5 is a flowchart diagram illustrating an exemplary method for treating sleep apnea (e.g., using the systems described herein).

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of embodiments according to the present disclosure will now be presented with reference to various apparatus and methods. These apparatus and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more example embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

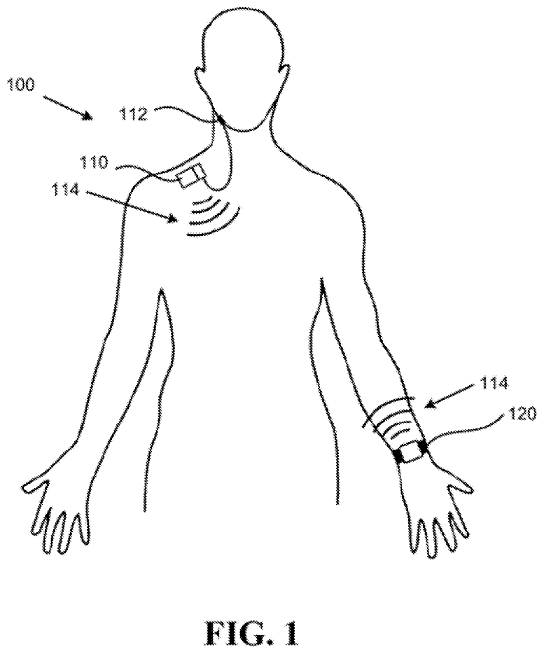

FIG. 1 is a diagram illustrating an embodiment of a system 100 for treating obstructive sleep apnea. In this example, a stimulator 110 is configured to be implanted within a patient. The stimulator 110 may be coupled to an electrode 112 (e.g., via an implantable lead). Although one electrode is shown in FIG. 1, in some embodiments two bilateral electrodes are used. The electrode 112 may be configured to be implanted within the patient, positioned to stimulate nerves which innervate an upper airway muscle. In some embodiments, the nerve is the hypoglossal nerve. In some embodiments which include two bilateral electrodes, each electrode may be configured to be implanted within the patient and positioned to stimulate a respective branch of the hypoglossal nerve (e.g., one electrode for the left branch, one electrode for the right branch). In some embodiments, the electrode 112 is a nerve cuff electrode. The system 100 may include one or more external devices, which may each comprise at least one external sensor 120 (e.g., a body-worn sensor). For example, the external device may be a smartphone (e.g., an Apple® iPhone® or an Android® based device), a smart watch, or a fitness or activity watch, or a monitor. In some aspects, the external device may be a housing configured to include and/or communicate with an external sensor 120 and a communication system capable of wired or wireless communication with one or more components of the system 100 described herein. The external device may be in wireless communication with the stimulator 110 via a wireless link 114. For example, the wireless link 114 may be Bluetooth, or another wireless communication protocol (e.g., the wireless link 114 may use a frequency within the FCC-regulated MedRadio band, 401-406 MHz).

The stimulator 110 may comprise or be in communication with at least one internal sensor implanted into a patient, such as a pressure sensor or inertial measurement unit (IMU) configured to generate sensory data corresponding to the movement of the thoracic or abdominal cavity of a patient during respiration. Each external sensor 120 may comprise at least one physiological sensor configured to generate sensory data corresponding to a physiological parameter of a patient wearing (or in close proximity to) the external sensor 120 (e.g., as a ring, watch, or headband, or held against or in proximity to the body in a harness). For example, an external sensor 120 may comprise a sensor configured to generate sensory data corresponding to the peripheral oxygen saturation (SpO₂), heart rate, heart rhythm, heart rate variability, and/or respiration. In some aspects, an external sensor 120 may be a sensor configured to generate electrocardiogram (ECG), electroencephalogram (EEG), or electrooculogram (EOG) data. Electrode contacts in an external sensor that are in contact with the skin can be either of the dry or wet type.

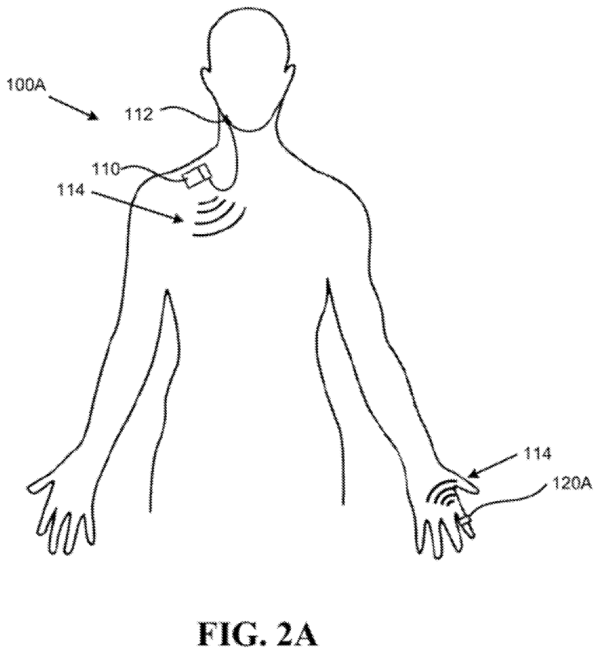

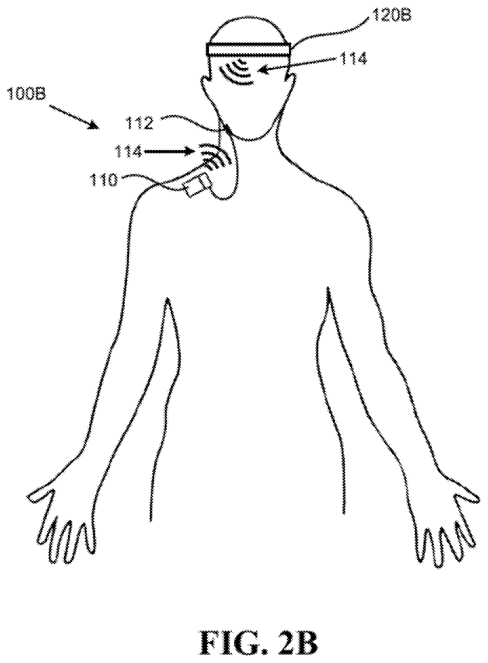

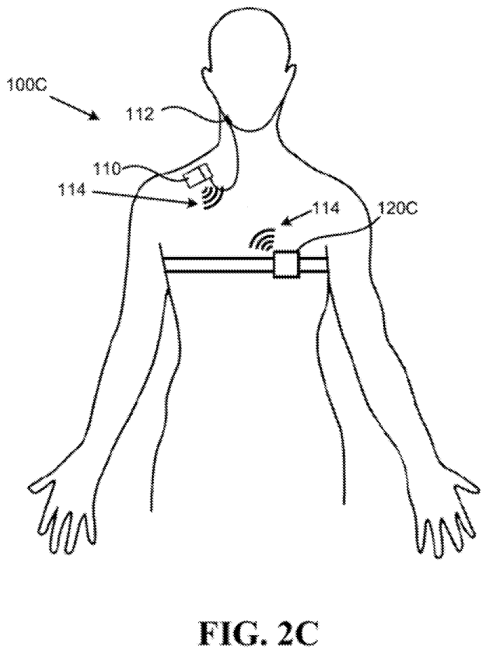

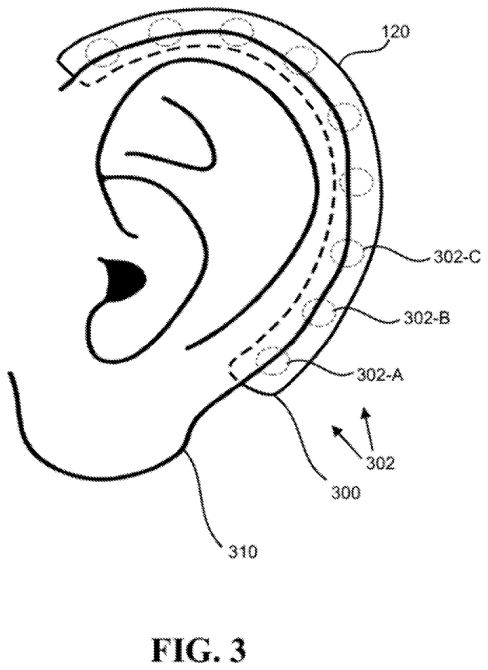

An external sensor 120 in wireless communications with the stimulator 110 may take various forms. In some embodiments, as shown in the exemplary embodiment depicted in FIG. 1, an external sensor 120 may be configured to be worn on or around a wrist of a patient. For example, the external sensor 120 may be an element of, a watch, a smart watch, or a fitness monitor. Other exemplary embodiments will be described with respect to FIGS. 2A-C and FIG. 3.

FIG. 2A is a diagram illustrating an embodiment of a system 100A for treating obstructive sleep apnea with a finger-mounted external sensor 120A. An external sensor 120A may be configured to be worn on or around a finger of a patient (e.g., a ring type sensor). An external sensor 120A may be configured to sense SpO₂, heart rate, heart rhythm, and/or heart rate variability.

FIG. 2B is a diagram illustrating an embodiment of a system 100B for treating obstructive sleep apnea with a head-mounted external sensor 120B. An external sensor 120B may be configured to be worn on or around a head of a patient. For example, the external sensor 120B may be, or may be incorporated into, a headband, hat, cap, or other suitable headwear. An external sensor 120B may be configured to sense EEG and/or EOG signals.

FIG. 2C is a diagram illustrating an embodiment of a system 100C for treating obstructive sleep apnea with a chest-mounted external sensor 120C. An external sensor 120C may be configured to be worn around a chest of a patient. For example, the external sensor 120C may be, may be incorporated into, or may be attached to the patient using a chest band. The external sensor 120C may be configured to sense ECG, heart rate, heart rate variability, heart rhythm, and/or respiration.

FIG. 3 is a diagram illustrating an embodiment of an EEG sensor 300 shaped and configured for positioning behind the ear 310 of a patient. A system for treating obstructive sleep apnea may include the EEG sensor 300 as an external sensor 120. The EEG sensor 300 includes at least two electrode contacts 302 configured to contact the skin behind a patient's ear when worn by the patient. The EEG sensor 300 may take measurements (e.g., voltage measurements) at the two electrode contacts 302 to generate EEG data.

In some embodiments, the EEG sensor 300 may include more than two electrode contacts 302-A, 302-B, 302-C, etc. The EEG sensor 300 may be configured to use the more-than-two electrode contacts to select at least two electrode contacts out of the group of contacts 302 with the best EEG signal available, and may be configured to use the selected electrode contacts to generate EEG data for processing and communication to the system via the wireless link. In some embodiments, the EEG sensor 300 may be configured to evaluate EEG measurements gathered using respective pairs of electrode contacts and may select the electrode contacts to use for generating the EEG data based on the evaluation. In some aspects, the EEG sensor 300 may transmit EEG measurements gathered using respective pairs of electrode contacts to another device via the wireless link (e.g., to the stimulator 110, or to a remote control 130 or a clinician programmer 140, described further below), and the other device may select the electrode contacts to use for generating the EEG data and may transmit an identifier of the selected electrodes to the EEG sensor 300.

In some aspects, a system for the treatment of OSA may include multiple EEG sensors per patient (e.g., one EEG sensor 300 for each ear of the patient). The multiple EEG sensors may be used to provide multiple streams of sensory data to the stimulator 110. Similarly, multiple ECG or EOG sensors per patient may be used to provide multiple streams of sensory data for analysis by the stimulator 110.

In some aspects, a system for the treatment of OSA may include a plurality of external sensors 120. Each external sensor may be configured to generate a different form of sensory data regarding physiological parameters of the patient. For example, each external sensor may independently comprise an inertial sensor, a pressure sensor, an audio sensor, an LED-based SpO₂ sensor), etc.

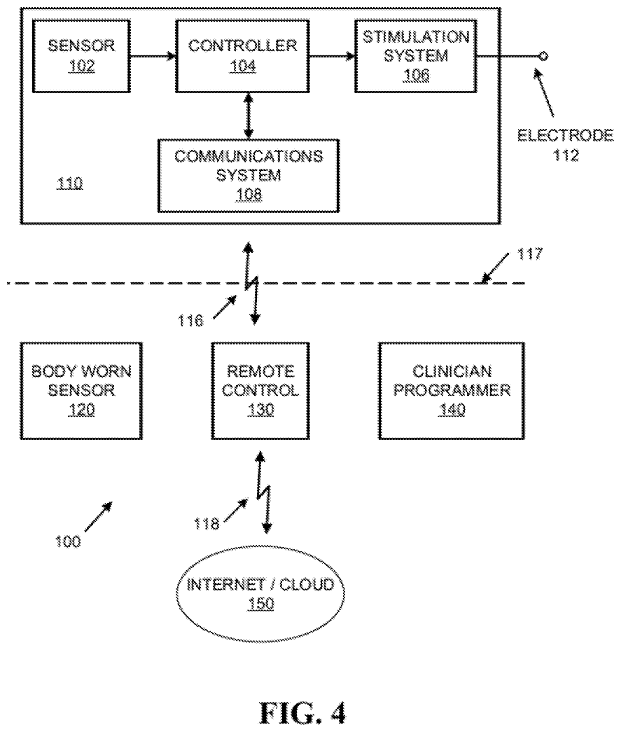

FIG. 4 is a block diagram of an embodiment of a system 100 for treating obstructive sleep apnea. It includes an implantable stimulator 110, an external sensor 120, a remote control 130, and a clinician programmer 140. In this example, the stimulator 110 includes a sensor 102, a controller 104, a stimulation system 106, an electrode 112, and a communications system 108.

The sensor 102 may be configured to generate sensory data corresponding to the respiration of the patient, and to transmit the sensory data to the controller 104. The stimulation system 106 (e.g., an implantable pulse generator, “IPG”) may be configured to apply stimulation to the electrode 112. The controller 104 may be configured to control when and/or how the stimulation system 106 applies stimulation to the electrode 112. The controller 104 may be configured to determine the respiratory waveform of the patient or a model of the respiratory waveform based at least in part on the sensory data from the sensor 102 and to control the simulation system 106 based on the respiratory waveform or model respiratory waveform. For example, the controller 104 may control the stimulation system 106 to apply stimulation during the inspiratory period of the respiration waveform, to apply stimulation during the expiratory period of the respiratory waveform, or to apply stimulation during particular parts of the inspiratory and/or expiratory portions of the respiratory waveform.

The communications system 108 may provide one or more wireless links 116, through the skin 117 of a patient, to at least one external sensor 120, the remote control 130, and/or the clinician programmer 140. The external sensor 120, the remote control 130, and the clinician programmer 140 may also include respective communications systems, which may provide wireless links 118 between the external sensor 120, the remote control 130, the clinician programmer 140, and the Internet and/or cloud services 150. The wireless links 116 and/or 118 can utilize Bluetooth, Bluetooth Low Energy, or other wireless communication protocols. The wireless links 116 and/or 118 may include authentication and encryption suitable to protect patient data.

In some embodiments, at least one sensor 102 is a pressure sensor or an inertial measurement unit (IMU) configured to generate sensory data corresponding to the movement of the thoracic or abdominal cavity of a patient during respiration. In one embodiment, the sensor 102 is a 6-axis IMU. The IMU data may be used to extract various physiological parameters related to respiration, such as respiration rate and/or amplitude. In some embodiments, the IMU sensor may sense a seismo-cardiogram (SCG), which is the mechanical wave caused due to the heartbeat of the patient. The controller 104 may process the SCG data received from the IMU sensor to extract various features, such as heart rate and heart rate variability. These features can be used by the controller to aid in the detection of the respiration pattern of the patient. In some embodiments, the controller 104 may apply a system of machine learning algorithms to the features to aid in detection of the respiration pattern. The sensory data from the IMU can be the primary source of data for the controller 104 to determine the appropriate stimulation timing or parameters to be given to the stimulation system 106 and to the nerve being stimulated via the electrode 112.

The system 100 may be configured to deliver stimulation to a nerve innervating the upper airway of the patient through the electrode 112 implanted proximate the nerve. In some embodiments, the nerve is the hypoglossal nerve. In some embodiments, the upper airway muscle comprises the genioglossus, the geniohyoid, or some combination thereof. When the nerve is stimulated, it activates the upper airway muscle, thereby preventing or alleviating obstructive apneic events. In some embodiments, the stimulation system 106 applies stimulation to the nerve with an intensity sufficient to promote tonus in the upper airway muscle. In some embodiments, the stimulation system 106 applies stimulation to the nerve with an intensity sufficient to cause bulk muscle movement in the upper airway muscle. The stimulation system 106 is coupled to controller 104. The controller 104 receives the sensory data from one or more internal sensors 102 and/or sensory data from one or more external sensor 120, and controls when the stimulation system 106 applies stimulation. In some embodiments, the controller 104 can control the intensity of the stimulation applied by the stimulation system 106. In some embodiments, the stimulation system 106 may apply different intensities of stimulation by changing the amplitude, the pulse width, or the frequency of the stimulation. In some aspects, the control 104 controls the amplitude, the pulse width, or the frequency of the stimulation applied by the stimulation system 106.

The stimulator 110 may be configured to receive sensory data from one or more external sensors 120 (e.g., a body-worn sensor) and to apply stimulation therapy to the patient based on the sensory data received from the external sensor 120. The external sensor 120 may have at least one physiological sensor configured to generate sensory data based on monitoring a physiological parameter of the patient, such as SpO₂, heart rate, or respiration, or based on sense ECG, EEG, or EOG data. SpO₂ data can be used to measure oxygen desaturation events, which could be used to provide additional data for apneic events. Apneic events can be detected by determining that the regular respiratory pattern, as detected by the implanted IMU or pressure sensor, has become irregular for a number of cycles (e.g., at least 1, 2, 3, 4, 5, 6, 7, 8, 9, or 10 cycles). Waveforms and parameters indicative of an irregular respiratory pattern are disclosed, e.g., in U.S. Pat. Nos. 5,540,731 and 8,938,299, the entire contents of which are incorporated herein by reference. Data obtained from the one or more external sensors 120 provides additional information that may be used (e.g., by the controller 104) for apnea detection, providing potential benefits such as increased accuracy and improved patient compliance. For example, an external LED-based SpO₂ sensor is less uncomfortable to a patient than a system that requires a CPAP mask. The external sensors 120 may take one of many forms such as: a watch or fitness monitor as shown in FIG. 1; a behind the ear EEG sensor as shown in FIG. 3; a ring type sensor worn on a finger for sensing SpO₂, heart rate, heart rhythm, heart rate variability as shown in FIG. 2A; a headband or head cap for sensing EEG or EOG signals as shown in FIG. 2B; or a chest band sensing ECG, heart rate, heart rate variability, rhythm and respiration as shown in FIG. 2C.

In some aspects, the system 100 may use the sensory data generated by at least one external sensor 120 to dynamically titrate therapy delivered to the patient, to determine when to apply stimulation to the patient, and/or when to turn stimulation therapy on or off. The controller 104 may utilize ECG or heart rate data received from the external sensor 120 to augment inertial data (either inertial data generated by the internal sensor 102, inertial data generated by the external sensor 120, or inertial data generated by a separate external sensor) to make sleep stage decisions which may be more accurate than sleep stage decisions based on inertial data alone. In some embodiments, the controller 104 may use machine learning methods to make the sleep stage decisions. The controller 104 may control the stimulation applied by the stimulation system 106 based on the sleep stage decisions. For example, the controller 104 may utilize the sleep stage decisions to determine whether to turn stimulation therapy on and off (e.g., turning stimulation therapy on when the sleep stage decisions indicate that the patient is asleep, turning stimulation therapy off when the sleep stage decisions indicate that the patient is not asleep).

In some aspects, the system 100 may use the sensory data generated by at least one external sensor 120 to determine parameters of the application of stimulation applied to the patient (e.g., where stimulation is applied during specific parts of the respiratory cycle, when to apply stimulation, and/or intensity of stimulation applied). For example, the external sensor 120 may comprise a respiration rate sensor. The controller 104 may utilize the respiration rate sensory data from the external sensor 120, in addition to or as an alternative to the sensory data from the internal sensor 102, to measure the respiratory cycle and control when stimulation is applied to the nerve during the respiratory cycle.

In some aspects, the system 100 may use the sensory data generated by at least one external sensor 120 to control when to turn therapy on or off. For example, an external sensor 120 that provides sleep stage and apneic event information can be used to turn on therapy at night and/or to dynamically titrate therapy based on the apnea-hypopnea index (AHI) which is an indication of the severity of a person's sleep apnea.

An external sensor 120 such as an EEG sensor may provide sleep stage data, i.e., alpha, theta, delta, sleep spindles and/or slow waves. One or more machine learning algorithms may classify the EEG data and generate probabilistic outputs for the various sleep stages. Such a system can be used to: quantify metrics of improvement of sleep quality, such as for example being able to measure OSA patients getting more REM sleep, with therapy on versus therapy off. Such a system may also dynamically titrate stimulation parameters based on the sleep stage, such as for example increasing pulse amplitude during REM stages when neural drive to the hypoglossal nerve is low. The system can also turn therapy on or off based on the detection of a patient's state of wakefulness. Sleep stage data can be periodically sent by the system 100 to the patient's physician or health care provider via the wireless connection 118 to the Internet 150. The statistical analysis of sleep metrics as a function of sleep stages for a patient or group of patients could prove to be useful to improve nerve stimulation algorithms for OSA patients.

In some aspects, the controller 104 may monitor the wireless link 116 between the external sensor 120 and the stimulator 110, and may apply stimulation therapy based on the wireless link 116. The controller 104 may be configured with a default therapy and with an external-sensor-influenced therapy which is responsive to the sensory data received from the external sensor 120. Where the wireless link between the external sensor 120 and the stimulation 110 is operational, the controller 104 may utilize the external-sensor-influenced therapy. In situations where the wireless link 116 becomes poor in quality, the controller 104 may utilize the default therapy to provide OSA treatment to the patient throughout the night. The stimulator 110 may thereby continue to provide OSA treatment when a poor wireless connection is experienced or where operation of the external sensor 120 fails (e.g., a battery of the external sensor 120 dies). The external sensor 120 may thus be used to “augment” decisions and not be used as the main sensor for timing stimulation. Such a system would therefore provide potentially better treatment to the patient in case the wireless link between the external sensors and the implanted stimulator 110 is inadequate for stable wireless communication. The system 100 may default to a safe mode when the wireless link fades or is not existent.

In some aspects the functionality provided by the controller 104 may be supplemented by an auxiliary controller (e.g., implemented as software on an external device that is wirelessly coupled to the controller 104 and optionally to one or more of the sensors 120). In such configurations, the implanted controller 104 would operate as the primary controller of the stimulation system 106 for safety purposes. However, processing of sensor data may be at least partially offloaded to the auxiliary controller. For example, computationally-intensive processing tasks such as machine learning based analysis of sensor data may be handled by the auxiliary controller. The output from such processing, such as timing and/or other intensity (e.g., voltage, frequency) parameters for stimulation, may then be communicated to the controller 104. The controller 104 may optionally be configured to take this additional input from the auxiliary controller into account when determining final parameters to be applied by the stimulation system 106. Offloading at least some processing to an auxiliary controller may advantageously conserve battery power. In addition, algorithms that would not have been considered viable due to the power constraints associated with an implanted battery may be reasonable to implement using an external device. Moreover, it is easier to update the firmware, software, etc. of an external device rather than an implanted device, and so an externally-situated auxiliary controller may be updated more frequently and/or easily.

The remote control 130 may communicate with the stimulator 110 to control aspects of the operation of the stimulator 110 based on user input received at the remote control 130. For example, the remote control 130 may be configured to receive a user input identifying a selected intensity for treatment. The remote control 130 may communicate the selected intensity to the stimulator 110 via the communications system 108, and the controller 104 may control the intensity of therapy applied based on the selected intensity. In another example, the remote control 130 may be configured to receive a user input selecting an on/off state for the system 100. The remote control 130 may communicate the selected on/off state to the stimulator 110 via the communications system 108, and the controller 104 may control whether therapy is applied by the stimulator 110 based on the selected on-off state. The clinician programmer 140 may be configured to receive user input (e.g., from a clinician configuring the stimulator 110) and to transmit the user input to the stimulator 110 via the communications system 108. The user input received from the clinician programmer 140 may be configuration information for operation of the stimulator 110 (e.g., identifying contacts of a multi-contact electrode to which stimulation should be applied; identifying an intensity of stimulation to be applied or a range of allowed intensities), and the controller 104, the stimulation system 106, or another element of the simulator 110 may operate based on the received configuration information. The remote control 130 and/or the clinician programmer 140 may be implemented using a smartphone, tablet, or other computing device configured with an application for communicating with the stimulator 110.

In some embodiments, the Internet and/or cloud services 150 may provide a history related to OSA treatment for the patient. For example, the stimulator 110 may transmit data related to therapy applied (e.g., duration of applied stimulation or intensity of applied stimulation) or related to efficacy of treatment (e.g., apnea-hypopnea index (AHI)) to the remote control 130 or to the clinician programmer 140, and the remote control 130 or the clinician programmer 140 may transmit the data to the Internet and/or cloud service 150 to be compiled. In some embodiments, the Internet and/or cloud services 150 may provide for remote monitoring of OSA treatment for the patient. For example, the data related to therapy applied or related to efficacy of treatment may be compiled and made accessible to a doctor or clinician providing OSA treatment for the patient. In some aspects, the stimulator 110 may transmit the data related to therapy applied or related to efficacy of treatment to the remote control 130, the remote control 130 may transmit the data to the Internet and/or cloud services 150, and the clinician programmer 140 may access and display the compiled data to assist the user of the clinician programmer 140 in the configuration of the stimulator 110. In some embodiments, the Internet and/or cloud services 150 may provide for remote updating of OSA treatment for the patient. For example, the clinician or doctor may make configuration changes via the Internet and/or cloud services 150, and the Internet and/or cloud services 150 may transmit the configuration changes to the stimulator 110 via the remote control 130 or the clinician programmer 140.

In some aspects, the system 100 may further comprise an external power source (e.g., a rechargeable or primary battery pack) configured to power the at least one sensor 102, the stimulation system 106, and/or the communications system 108. Power may be provided to any implanted components of the system, by induction. An external battery pack can be easily replaced compared to an implanted device with a battery. No surgeries are required to replace the battery because the battery is a part of the external system. Systems configured according to this exemplary aspect are advantageous in that the use of an external power source allows for much smaller implantable systems and consequently, less invasive surgery. Furthermore, by moving the power source to an external location, size is no longer a limiting factor. Large battery packs can be used, allowing for systems that are capable of providing therapy for longer periods of time. Similar benefits are provided by the use of at least one external sensor 120. An implanted sensor has to be surgically implanted in location that allows for the sensing of one or more adequate respiration artifacts. In contrast, by placing the sensor externally to the body, the risk of poor placement is mitigated. Furthermore, external components (e.g., a power source and the one or more external sensors 120) may be more easily updated (e.g., to update firmware, or to adjust algorithms used by such devices) and in the case of external sensors 120, data may be more easily exported from such devices (e.g., for diagnostic purposes or for clinical studies).

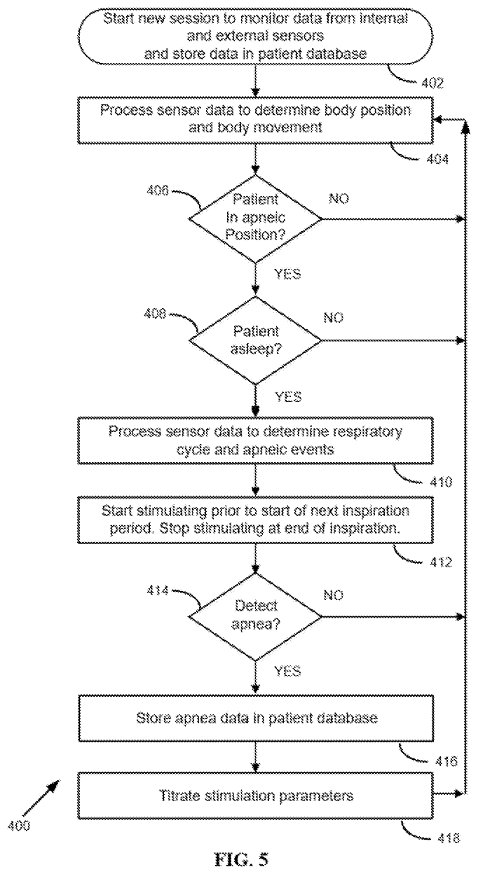

FIG. 5 is a flowchart diagram illustrating an exemplary method for treating sleep apnea, e.g., using the systems described herein. As illustrated by FIG. 4, such a method may begin (in this example at step 402) by starting new session to monitor data received from the one more internal sensors 102 and external sensors 120 described herein. This session may be implemented, e.g., in software running on the controller 104. The controller may be configured to begin a session, e.g., upon manual activation by a patient (e.g., using a remote control or application), triggered upon the detection of one or more parameters from the internal sensor 102 and/or external sensors 120 (e.g., upon detection that a patient is asleep). In some aspects, the session may be timed to begin after a predetermined amount of time has elapsed following manual activation or detection of triggering parameters (e.g., 5, 10, 15, 20, 25, or 30 minutes after either event). This sensor data is then processed (in this example at step 404) to determine one or more parameters that can be used to determine whether a patient is in an apneic position, such as the body position and body movement, and whether the patient is asleep. These activities are illustrated by steps 406 and 408 in this example. If the patient is determined to be awake and/or not in an apneic position, monitoring may continue without the need for a therapeutic intervention, as shown here by the flowchart returning to step 404 when either of these conditions is not detected. Alternatively, if the patient is in an apneic position and asleep, sensor data may be processed to determine the respiratory cycle and whether any apneic events have occurred. Stimulation may be applied, e.g., after the patient has returned to a stable respiratory cycle following a detected apneic event. In some aspects, once the pattern of a patient's respiratory cycle while asleep is detected, stimulation may be timed to start prior to the start of the next inspiration period and configured to stop at the end of inspiration of the next respiratory cycle. The timing and parameters of stimulation (e.g., voltage and frequency) can be adjusted during the titration step 418. In some aspects, adjustments may be based upon data received from the internal sensor 102 and/or external sensors 120. The timing and parameters of stimulation may also be selected based upon an assessment of the patient's sleep state. Less aggressive therapy may be applied during light sleep, compared to if a patient is determined to be in deep sleep or REM sleep. If apnea is detected, in some aspects the apnea data may optionally then be stored in a patient database, as shown by step 416. This data may optionally be used to titrate stimulation parameters as monitoring continues, as shown by step 418. A monitoring session may be configured to end upon manual deactivation by the patient or upon a triggering condition. For example, the controller may be configured to end a session, upon manual deactivation by a patient (e.g., using a remote control or application), triggered upon the detection of one or more parameters from the internal sensor 102 and/or external sensors 120 (e.g., upon detection that a patient is awake, or in a sitting or standing position). In some aspects, the session may be timed to end after a predetermined amount of time has elapsed following manual deactivation or detection of triggering parameters (e.g., 5, 10, 15, 20, 25, or 30 minutes after either event). It is understood that the method illustrated by this flowchart is exemplary and non-limiting. Any of the steps shown in this workflow may be omitted in alternative aspects, and the order of the illustrated steps may also be adjusted as needed (e.g., one may perform alternative permutations of this method my carrying out any of the disclosed steps, in any order).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Terms such as “if,” “when,” and “while” should be interpreted to mean “under the condition that” rather than imply an immediate temporal relationship or reaction. That is, these phrases, e.g., “when,” do not imply an immediate action in response to or during the occurrence of an action, but simply imply that if a condition is met then an action will occur, but without requiring a specific or immediate time constraint for the action to occur. The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

What is claimed is:

1. A system for treating obstructive sleep apnea in a patient comprising:
at least one external sensor configured to sense a physiological parameter of the patient; and
a stimulator comprising:
an internal sensor configured to generate a first signal corresponding to movement of the thoracic or abdominal cavity of the patient during respiration;
a stimulation system configured to deliver stimulation to a nerve which innervates an upper airway muscle; and
a controller coupled to the internal sensor and the stimulation system, and wirelessly coupled to the at least one external sensor;
wherein the controller is configured to measure the respiratory cycle of the patient based on the first signal and the sensed physiological parameter, and to cause the stimulation system to stimulate the nerve based on the measured respiratory cycle.

2. The system of claim 1 wherein the at least one external sensor provides at least one of: peripheral oxygen saturation (SpO₂) data, heartrate, heart rate variability, heart rhythm, respiration, electrocardiogram (ECG) data, electroencephalogram (EEG) data, or electrooculogram (EOG) data.

3. The system of claim 1, wherein the at least one external sensor comprises at least one of: a wrist wearable watch, a wrist wearable activity tracker, a ring sensor configured to be worn on a finger, a headband, a head cap, a chest band, or a behind the ear sensor.

4. The system of claim 1, wherein the system comprises a plurality of external sensors.

5. The system of claim 1, wherein the system comprises a plurality of external sensors, each configured to sense a different physiological parameter of the patient.

6. The system of claim 1, wherein the stimulator further comprises an auxiliary controller comprising software executed on an external device, wherein the auxiliary controller is wirelessly coupled to the controller, and optionally to the at least one external sensor, and configured to process data received from the controller and/or the at least one external sensor.

7. The system of claim 6, wherein the auxiliary controller is configured to process data received from the controller and/or the at least one external sensor by:
determining timing and/or intensity parameters for stimulation based on the received data using at least one machine learning algorithm; and
communicating the timing and/or intensity parameters to the controller.

8. The system of claim 1 wherein the controller is configured to cause the stimulation system to stimulate the nerve during the inspiratory portion of respiration; during the expiratory portion of respiration; or during the inspiratory portion and the expiratory portion of respiration.

9. The system of claim 1 wherein the stimulator is implantable.

10. The system of claim 1 wherein the internal sensor is at least one of: an inertial measurement unit or a pressure sensor.

11. The system of claim 1 wherein the at least one external sensor comprises an inertial sensor, a pressure sensor, an audio sensor, a SpO₂ sensor, or a sensor configured to detect a RIP (Respiratory Inductance Plethysmography) band.

12. A method of treating obstructive sleep apnea in a patient comprising:
acquiring a first set of sensory data from an implanted sensor corresponding to movement of the thoracic or abdominal cavity of the patient during respiration;
acquiring a second set of sensory data via a wireless link from at least one external sensor, the second sensory data corresponding to one or more physiological parameters of the patient;
determining a respiratory cycle of the patient based on the first and second sets of sensory data; and
stimulating a nerve innervating an upper airway muscle during a stable respiratory cycle following an apneic event, wherein apneic events are determined based on the respiratory cycle of the patient.

13. The method of claim 12 further comprising identifying an inspiratory portion of the respiratory cycle.

14. The method of claim 13 wherein stimulating the nerve innervating an upper airway muscle is stimulating the nerve during the inspiratory portion of the respiratory cycle.

15. The method of claim 12 wherein the at least one external sensor provides at least one of: peripheral oxygen saturation (SpO₂) data, heartrate, heart rate variability, heart rhythm, respiration, electrocardiogram (ECG) data, electroencephalogram (EEG) data, or electrooculogram (EOG) data.

16. The method of claim 12 wherein the at least one external sensor comprises at least one of: a wrist wearable watch, a wrist wearable activity tracker, a ring sensor configured to be worn on a finger, a headband, a head cap, a chest band or a behind the ear sensor.

17. The method of claim 12, wherein the internal sensor is at least one of: an inertial measurement unit or a pressure sensor.

18. The method of claim 12, wherein the second set of sensory data is acquired from a plurality of external sensors.

19. The method of claim 18, wherein each external sensor is configured to detect sensory data corresponding to a different physiological parameter of the patient.

20. The method of claim 12, wherein one or more parameters for stimulating the nerve innervating the upper airway muscle are based on the respiratory cycle of the patient.

21. The method of claim 20, wherein the parameters comprise a timing and/or an intensity of the stimulation.

22. The method of claim 12, further comprising:
determining a sleep state of the patient based on the first and/or second sets of sensory data;
wherein one or more parameters for stimulating the nerve innervating the upper airway muscle are based on the sleep state of the patient.

23. The method of claim 22, wherein determining the sleep state comprises determining whether the patient is in light sleep, deep sleep, or REM sleep, and the parameters comprise a timing and/or an intensity of the stimulation.
